(19) United States
(12) Patent Application Publication
(10) Pub. No.: US 2007/0093755 A1
(43) Pub. Date: Apr. 26, 2007

(54) Cannula handle and storage system

(76) Inventors: David R. Koos, San Diego, CA (US); Brian F. Pockett, San Marcos, CA (US); Philip Watts, Long Beach, CA (US)

Correspondence Address: THE LAW OFFICE OF JOHN A. GRIECCI, 703 PIER AVE., SUITE B #657, HERMOSA BEACH, CA, 90254, US

(21) Appl. No.: 11/527,013

(22) Filed: Sep. 25, 2006

Related U.S. Application Data
(60) Provisional application No. 60/720,339, filed on Sep. 23, 2005.

(51) Int. Cl. A61M 1/00; A61M 5/178

(52) U.S. Cl. 604/164.01; 604/35; 604/164.07

(57) ABSTRACT

A cannula, handle and suction device to provide for the withdrawal of fluids from a body. A handle shaft has a receptacle connects to a cannula tapered bore leading to an end of a cannula shaft. The receptacle has a post that is conformingly received by the tapered bore such that an end of the post abuts the end of the cannula shaft. The cannula shaft has lubrication over its exterior. The handle shaft has an inner layer composed of a material that is destroyed when autoclaved, and a layer of lubricant covering an inner surface of the shaft. The handle shaft forms a chamber that may be sealed with a closure, and the chamber may opens into the channel such that the chamber acts as a Helmholtz resonator. The handle has a removable grip with a diameter of at least one inch and a circumferential flange.

This application claims the benefit of U.S. provisional Application No. 60/720,339, filed Sep. 23, 2005, which is incorporated herein by reference for all purposes.

This invention relates generally to preferably disposable tissue-transfer devices such as cannula, syringes and connectors, and more particularly, to handles and tissue storage containers for a cannula.

### BACKGROUND

Typically, a cannula is provided with a simple handle integral with the cannula at a proximal end of the cannula. The handle is typically a cylindrical body having a diameter of approximately ½ to ⅞ inch. During liposuction, a medical practitioner is required to repeatedly move the cannula in and out of the patient's body through a small orifice in the body. The practitioner may experience significant resistance to this longitudinal motion, and therefore, the handle must be strong, and it is not uncommon for a practitioner to experience significant fatigue of the hand and forearm. Moreover, typical handles are metal, which may provide for reusability at the expense of weight.

To remove tissue through a cannula, some type of vacuum-generating equipment is used. For the removal of a small quantity of tissue, the vacuum-generating device may be a syringe, or it may be a separate, powered vacuum pump. For large volume aspiration, which can be on the order of one to two leaders of tissue, a pump is commonly used. In some cases, cannula are used to remove tissue intended for storage or reuse, such as for the extraction of stem cells. Such tissue may be fairly sensitive, and subject to damage from rough handling, such as by passing through a pump.

When a cannula is used to draw tissue from a human body, the tissue is typically received into the cannula in short bursts. A strong grip on a cannula handle is normally necessary to overcome the force of the abrupt cannula movements in response to these bursts. Moreover, the resulting sudden jerking of the cannula causes significant user fatigue, and potentially may cause bruising or other damage to the patient.

When a syringe is used to create vacuum for liposuction, the cannula and syringe must have a connection system (i.e., mating connectors). Typically, the cannula is provided with an adaptor configured to mate with a structure (e.g., a receptacle) on the syringe. The receptacle is configured with a passageway to connect an interior cavity of a syringe barrel to a channel through the cannula. A common form of receptacle used to connect medical components, and place them in fluid communication, is a “LUER LOK”® receptacle. such receptacles come in many sizes, and many commonly used syringes are configured with these receptacles.

A LUER LOK® receptacle includes a cylindrical hub having an outer cylindrical surface that is smooth, and an inner cylindrical surface threaded with double lead female threads. The hub extends from an inner end affixed to, or unitary with, the syringe, to an outer end. The receptacle further includes a tapered post extending concentrically through the hub. The tapered post extends from an inner end affixed to, or unitary with, the syringe, to an outer end extending beyond an outer end of the hub. The tapered post defines an outer surface with a circular cross-section, the diameter of which tapers uniformly from a larger size at the inner end of the tapered post to a smaller size at the outer end of the tapered post. The tapered post further defines an inner cylindrical surface, concentric with the outer surface and the hub. The inner surface forms a passageway through the tapered post. This passageway extends from an inner end that is in fluid communication with the interior of the syringe barrel, to an outer end that provides an opening to be placed in fluid communication with the cannula.

Existing adapters configured to connect to such a receptacle are described throughout U.S. Pat. Nos. 5,002,538, and 6,569,118, which are incorporated herein by reference for all purposes. Typically, these adapters are characterized by having a fitting that receives the end of a medical device such as a cannula. The cannula end is provided with an opening that is to be placed in fluid communication with the inside of the syringe barrel. The fitting is configured to be screwed into the hub of the receptacle using the threads on the hub. The fitting is further configured to hold the cannula end opposing the tapered post of the receptacle, such that fluid can flow between the medical device and the passageway through the tapered post. The fitting defines a tapered bore for receiving the tapered post. The tapered bore of the fitting tapers down to a size smaller than the outer diameter of the tapered post at its outer end. As a result, when the fitting is screwed into the threads of the hub, the outer surface of the tapered post conforms to and squeezes against the tapered bore of the fitting, thus forming a seal to prevent the flow of fluid and gas. To provide for that seal to be tight, the fitting is designed to create the seal with the end of the cannula shaft and the tapered post still a distance apart, thus providing a zone between the two in which fluid flow is disturbed rather than smooth.

Using the above-described device, sensitive withdrawn fluids (e.g., tissue) can be disturbed by contact with the material of the cannula shaft, by high levels of suction, and by discontinuous flow through the syringe/cannula interface.

Accordingly, there has existed a need for a handle and cannula to provide for the withdrawal of fluids, with a minimum of disturbance to the fluid, with a minimum of difficulty for a user, and with a minimum of distress to the body from which the fluids are being withdrawn. Furthermore, there is a need for such a device to be both reliable and easy to use, and for control over whether the device is reused. Preferred embodiments of the present invention satisfy these and other needs, and provide further related advantages.

### SUMMARY

In various embodiments, the present invention solves some or all of the needs mentioned above, providing a handle, cannula and suction device to provide for the withdrawal of fluids from a body, with a minimum of disturbance to the fluid, with a minimum of difficulty for a user, and with a minimum of distress to the body from which the fluids are being withdrawn, that are preferably reliable and easy to use, offering control over whether the devices are reused.

In one form, the invention provides a handle for use with a cannula forming a cannula channel, and a vacuum source forming a vacuum-source channel. The vacuum source has a receptacle including a hub with an outside cylindrical surface and a concentric threaded inside cylindrical surface. The vacuum source further has a post with a tapered outer surface concentric within the hub, the post forming a passageway concentrically within the post. The passageway forms an opening at an outer end of the post, the passageway being in fluid communication with the vacuum-source channel.

The handle includes a connector configured for connection to the cannula, a shaft forming a handle channel, the shaft and connector being configured to place the handle channel in fluid communication with the cannula channel, and a fitting attached to the shaft. The fitting includes an end portion forming a tapered bore in fluid communication with the handle channel, the tapered bore leading to an end of the shaft. The fitting end portion further forms a concentric outer threaded surface configured for threadedly receiving the threaded inside cylindrical surface of the hub. The tapered bore is configured to conformingly receive the tapered outer surface of the receptacle post as the fitting outer threaded surface threadedly receives the threaded inside cylindrical surface of the hub.

The tapered bore is sized to allow the outer end of the post to compressively contact the end of the shaft. Advantageously, this provides for the outer end of the post and the end of the handle shaft to form a first seal therebetween, where the seal operates to minimize the disturbance of fluid flowing between the cannula and the handle. The invention may further provide for an elastic sealing device configured to be compressed between the fitting and the receptacle when the receptacle post is received within the tapered bore, to add an additional seal between the fitting and the receptacle.

In another form, the invention provides a handle for use with a cannula forming a channel, and a vacuum source. The handle includes a connector configured for connection to the cannula, a shaft having an inner surface forming a handle channel, the shaft and connector being configured to place the handle channel in fluid communication with the cannula channel, and a fitting attached to the shaft, the shaft and fitting being configured to place the handle channel in fluid communication with the vacuum source. Advantageously, this form of the invention further provides a layer of lubricant covering the inner surface of the shaft to minimize disturbance of the fluid flowing through the shaft.

In another form, the invention also provides a handle for use with a cannula forming a channel, and a vacuum source. The handle has a connector configured for connection to the cannula, and a shaft having an inner layer forming a handle channel. The shaft inner layer is composed of a material that will cause the inner layer to be destroyed (e.g., significantly distorted) when autoclaved, thereby advantageously preventing reuse of a handle not intended for reuse.

Another form of the invention provides a handle for use with a cannula forming a channel, and a vacuum source. The handle includes a shaft having an inner surface forming a handle channel, a distal connector on a distal end of the shaft, the distal connector being configured for connection to the cannula, and a proximal connector on a proximal end of the shaft, the proximal connector being configured for connection to the vacuum source. The shaft, distal connector and proximal connector are configured to place the cannula channel in fluid communication with the vacuum source via the handle channel. To provide for ease of use, the shaft advantageously forms a circumferential surface for grasping by a user, the circumferential surface having a diameter of at least one inch.

Another form of the invention provides a handle for use with a cannula forming a channel, and a vacuum source, the handle including a shaft having an inner surface forming a handle channel, a distal connector on a distal end of the shaft, the distal connector being configured for connection to the cannula, and a proximal connector on a proximal end of the shaft, the proximal connector being configured for connection to the vacuum source. The shaft, distal connector and proximal connector are configured to place the cannula channel in fluid communication with the vacuum source via the handle channel. The shaft forms a grip configured for grasping by a user, and further forms a first circumferential flange extending radially outward from the grip by at least one-half inch, providing for ease of use and maximum control over the cannula to minimize the effects of cannula jerking on a user and/or a patient.

The invention, in another form, includes a handle for use with a cannula forming a channel, and a vacuum source. The handle has a shaft having an inner surface forming a tissue storage chamber, a first connector forming a first passageway, the first connector being configured for connection to the cannula, and a second connector forming a second passageway, the second connector being configured for connection to the vacuum source. The shaft, the first connector and the second connector are configured to place the cannula channel in fluid communication with the vacuum source via the tissue storage chamber. Advantageously, the tissue storage chamber preferably includes a portion having a diameter larger than that of the first and second passageways, and the handle is preferably formed of a distal portion including the first connector and a proximal portion including the second connector, the first and second portions being separably attached, and the first and second portions being detachable to provide direct access to the tissue storage chamber. The handle preferably further includes a first channel closure configured to seal tissue within the tissue storage chamber with respect to at least one of the cannula and the vacuum source.

In another form, the invention includes a handle for use with a cannula forming a channel, and a vacuum source. The handle includes a body having a first inner surface forming a tissue storage chamber and a second inner surface forming a channel that bypasses the tissue storage chamber. The handle further includes a first connector forming a first passageway, the first connector being configured for connection to the cannula, and a second connector forming a second passageway, the second connector being configured for connection to the vacuum source, wherein the channel places the first and second passageways in fluid communication. Advantageously, the tissue storage chamber opens into the channel, such that the chamber can act as a form of Helmholtz resonator.

In yet another form, the invention includes a handle for use with a cannula forming a channel, and a vacuum source. The handle includes a handle body having an inner surface forming a handle channel, a distal connector on a distal end of the handle body, the distal connector being configured for connection to the cannula, and a proximal connector on a proximal end of the handle body. The proximal connector is configured for connection to the vacuum source, and the handle body, distal connector and proximal connector are configured to place the cannula channel in fluid communication with the vacuum source via the handle channel. Advantageously, this form of the invention further includes a removable grip that is conformingly received over the handle body.

The embodiments described below are provided with a number of novel concepts, each of which may separately be an aspect of the invention. Among the purposes addressed by some of these aspects, is to minimize hand and arm fatigue in medical practitioners during tissue removal; to create fluid tight seals between system components; to provide for undisturbed, smooth fluid flow through an interface between a cannula and a vacuum device; to provide a system for tissue capture with a minimum of tissue distress; to have fluid throwing through the cannula to be unaffected by the material of the cannula; to have fluid throwing through the cannula to be less affected by flow rate anomalies; to lessen undesired effects on a patient due to sudden jerking of a cannula during tissue flow rate variations, to provide for components of a cannula system to be cost-effectively manufactured, and thus be disposable; to cause self-destruction of a cannula upon any attempt to sterilize the cannula for reuse; to provide uniform lubrication over a cannula exterior for the cannula to be safely inserted into a patient; and to avoid exposing removed tissue to excessive disturbance due to the effects of suction.

### BRIEF DESCRIPTION OF THE DRAWINGS

FIG. 1 is a side cross-sectional view of a first embodiment of a cannula and handle under the invention.

FIG. 2 is a side cross-sectional view of a cannula adapter and a handle receptacle of the embodiment depicted in FIG. 1.

FIG. 3A is a side cross-sectional view of a cannula adapter of the embodiment depicted in FIG. 1, having a first additional seal.

FIG. 3B is a side cross-sectional view of a cannula adapter of the embodiment depicted in FIG. 1, having an alternative of the first additional seal depicted in FIG. 3A.

FIG. 3C is a side cross-sectional view of a cannula adapter of the embodiment depicted in FIG. 1, having a second alternative of the first additional seal depicted in FIG. 3A.

FIG. 4 is a side cross-sectional view of a cannula shaft of the embodiment depicted in FIG. 1.

FIG. 5 is a side cross-sectional view of a second embodiment of a handle under the invention.

FIG. 6 is a side cross-sectional view of a third embodiment of a handle under the invention, attached to a cannula and a syringe.

FIG. 7 is a side cross-sectional view of a first variation of a fourth embodiment of a handle under the invention.

FIG. 8 is a side cross-sectional view of a second variation of a fourth embodiment of a handle under the invention.

FIG. 9 is a side cross-sectional view of a first variation of a fifth embodiment of a handle under the invention.

FIG. 10 is a side cross-sectional view of a second variation of a fifth embodiment of a handle under the invention.

FIG. 11 is a side cross-sectional view of a first variation of a sixth embodiment of a handle under the invention.

FIG. 12 is a side cross-sectional view of a second variation of a sixth embodiment of a handle under the invention.

### DETAILED DESCRIPTION OF THE PREFERRED EMBODIMENTS

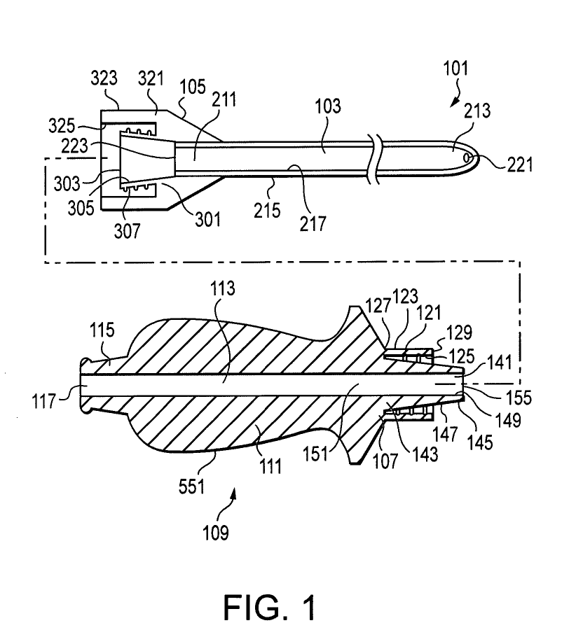

With reference to FIG. 1, a first embodiment of the invention includes a cannula 101 that includes a cannula shaft 103 and a proximal connector, e.g., a cannula adapter 105. The adapter is configured for use with a distal connector, e.g., a receptacle 107 of a handle 109, such as a LUER LOK® receptacle. This handle receptacle includes a shaft 111 that forms a channel 113, and a proximal connector, e.g., a handle vacuum line adaptor 115. The handle adaptor has a passageway 117 in fluid communication with the handle shaft channel, and is configured to receive a typical vacuum line (such as is used with a high-volume vacuum pump used for liposuction), and thereby place the handle adaptor passageway in fluid communication with the vacuum line.

As a result of this configuration, the handle is configured such that tissue received from a cannula passes from a distal end of the handle (i.e., through the handle receptacle), through the shaft of the handle, and potentially out a proximal end of the handle (i.e., through the handle adapter). Moreover, the handle may be provided to medical practitioners in a multitude of sizes, such that each practitioner may consistently use a hand-size independent cannula along with a handle that properly fits the practitioner's hand.

The cannula adapter 105 may be made from a metal, a plastic, a graphite material, a ceramic material, or a carbon fiber material. Optionally, the cannula adapter may be composed of a material having material properties such that the adapter is destroyed if the adapter is heated in an autoclave at temperatures necessary to sterilize medical instruments. As a result, the cannula will be a single use, disposable unit that cannot be reused.

Preferably, the handle receptacle includes a cylindrical hub 121 having an outer cylindrical surface 123 that is smooth, and an inner cylindrical surface 125 threaded with double lead female threads. The hub extends from an inner end 127 affixed to, or unitary with, the shaft, to an outer end 129. The receptacle further includes a tapered post 141 extending concentrically through the hub. The post extends from an inner end 143 affixed to, or unitary with, the shaft, to an outer end 145 extending beyond an outer end of the hub. The post defines a tapered, conical outer surface 147 with a circular cross-section, the diameter of which tapers uniformly from a larger size at the inner end of the tapered post to a smaller size at the outer end of the tapered post. The tapered post further defines an inner cylindrical surface 149, concentric with the outer surface and the hub, which forms a passageway through the post. This passageway extends from an inner end 151 that is in fluid communication with the interior channel 113 of the handle, to an outer end 155 at the post outer end 145, providing an opening to be placed in fluid communication with the cannula. The post outer end forms a flat, ring-shaped end-surface parallel to the surface of the opening.

The cannula shaft 103 is preferably composed of metal, such as is typically used for a cannula. Alternatively, the cannula shaft may be made from a plastic, a graphite material, a ceramic material, or a carbon fiber material. Optionally, the cannula shaft may be composed of a material having material properties such that the cannula shaft becomes destroyed if the cannula shaft is heated in an autoclave at temperatures necessary to sterilize medical instruments. As a result, the cannula will be a single use, disposable unit that cannot be reused.

The cannula shaft extends from a proximal end 211 to a distal end 213. The cannula shaft has a cylindrical outer surface 215, and a concentric cylindrical inner surface 217 that forms a channel extending between the proximal and distal ends. The distal end of the cannula is closed, but is provided with an opening 221 configured for the cannula to be used for the removal or in injection of fluids and/or tissue. The proximal end of the cannula shaft is open, and forms a flat, ring-shaped end-surface 223 conforming to the ring-shaped end-surface at the outer end 155 of the post 141.

The cannula adapter 105 is provided with a fitting 301 configured for attachment to the handle receptacle 107. The fitting is attached around the proximal end 211 of the cannula shaft 103. The fitting includes an end portion 303 forming a tapered bore 305 that is concentric with the cannula shaft channel, and that opens into the cannula shaft channel. The fitting end portion further forms an outer threaded surface 307 configured for threadedly receiving the threaded inside cylindrical surface 125 of the hub. The tapered bore is configured to conformingly receive the tapered outer surface of the receptacle post when the fitting outer threaded surface 307 threadedly receives the threaded inside cylindrical surface 125 of the hub 121. Unlike existing adapters, the tapered bore is sized such that the post may be inserted through the entire length of the tapered bore, allowing the longitudinal end at the outer end 145 of the post to press against the end of the cannula shaft. Thus, the ring-shaped end-surface of the post outer end may come in to concentrically aligned opposing contact with the ring-shaped end-surface at the proximal end of the cannula shaft.

The cannula adapter is further provided with an outer housing 321 concentrically surrounding the end portion 303 of the fitting 301. The housing defines a cylindrical outer surface 323 and a concentric cylindrical inner surface 325. The housing cylindrical inner surface approximately conforms to the outer surface 123 of the hub 121 when the adapter is received on the receptacle, while allowing enough clearance for the housing to be slid over the hub. More particularly, the clearance does not provide a press fit over the hub, and does not form a seal to prevent leakage from the interface between the cannula and the attached handle.

With reference to FIGS. 1 and 2, the cannula adapter 105 may be attached to the handle receptacle 107. More particularly, with the post 141 and the cannula shaft 103 concentrically aligned, the cannula adapter is slid over the handle receptacle such that the housing inner surface is received over the hub outer surface, and the post is received within the tapered bore of the fitting. The adapter is then rotated with respect to the handle such that the threads of the hub and the threads of the fitting threadedly receive each other, and such that the post advances further into the tapered bore of the fitting. The advance of the post into the tapered bore continues until the outer end of the tapered bore abuts the proximal end of the cannula shaft. Additional torque on the adapter frictionally locks the adapter in place relative to the handle, and presses the ring-shaped end-surface of the cannula shaft and post into a tightly abutting relationship.

Preferably, the cannula shaft channel has an inner diameter substantially the same as the inner diameter of the post passageway. Additionally, because the tapered surface of the post is conformingly received within the tapered surface of the fitting, the channel and the passageway are concentrically aligned such that tissue may flow between the channel and the passageway without any disturbance from significant variations or discontinuities along its flow path. Advantageously, this minimizes any damage that might occur to particularly sensitive fluids (e.g., tissue for stem cell cultures) that are being removed from a body.

In an alternative embodiment, the cannula channel may have an inner diameter over most of its length that is different than the diameter of the post passageway. For such a device, the proximal end of the cannula shaft preferably includes a gradual taper such that the inner diameter of the channel at the proximal end of the cannula shaft conforms to the inner diameter of the post passageway. Similar to a cannula shaft having an equal sized channel as its post's passageway, this minimizes any damage that might occur to particularly sensitive fluids due to discontinuities in their flow path.

The handle shaft channel 113 has an inner diameter substantially the same as the inner diameter of the post passageway, and preferably the same as the inner diameter of the cannula shaft channel. Moreover, preferably the handle adapter passageway 117 has an inner diameter substantially the same as the inner diameter of the cannula shaft channel. As previously noted, this minimizes any damage that might occur to particularly sensitive fluids (e.g., tissue for stem cell cultures) that are being removed from a body.

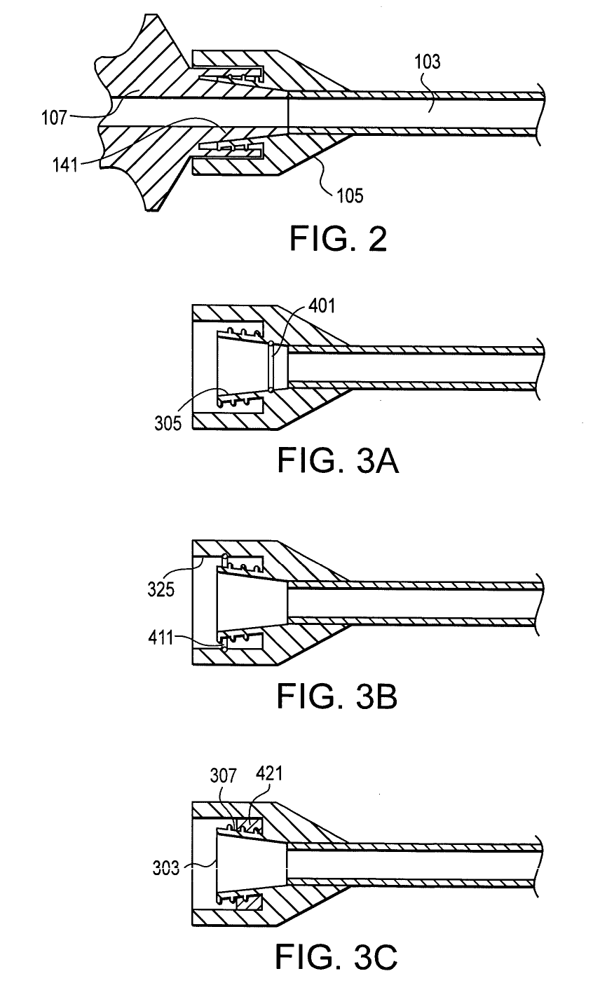

With reference to FIGS. 2 and 3A-3C, the adjoining end-surfaces of the post and the cannula shaft of an attached cannula and handle, form a first seal to limit the leaking of tissue from the cannula/handle, or the leaking of air into the cannula/handle. The interlaced threads of the fitting and hub provide a second seal to prevent leaking fluids or air.

Preferably an additional seal may be formed by using an elastic sealing device, such as an o-ring 401 received in a concentric groove around the diameter of the tapered bore 305 at a given longitudinal location within the tapered bore. Preferably, the o-ring is longitudinally close to the cannula shaft to minimize its distance from the first seal. When the post is fully inserted into the tapered bore, it passes through this tapered-bore o-ring, compressing the o-ring to form an additional seal.

Alternatively, preferably an additional seal may be formed by using an elastic sealing device, such as an o-ring 411 received in a concentric groove around the diameter of the housing inner surface 325 at a given longitudinal location within the housing. When the housing is received over the hub, the hub passes through this housing o-ring, compressing the o-ring to form an additional seal.

As a second alternative, preferably an additional seal may be formed by using an elastic sealing device, such as a gasket 421, received around the outer threaded surface 307 of the fitting end portion 303. When the threaded outer surface of the fitting is screwed into the threaded inner surface of the hub, the outer end of the hub presses into the gasket, forming an additional seal around the circumference of the fitting end portion.

Optionally, any or all of the above seal mechanisms may be combined into a single cannula adapter to provide for a more redundant and complete seal system.

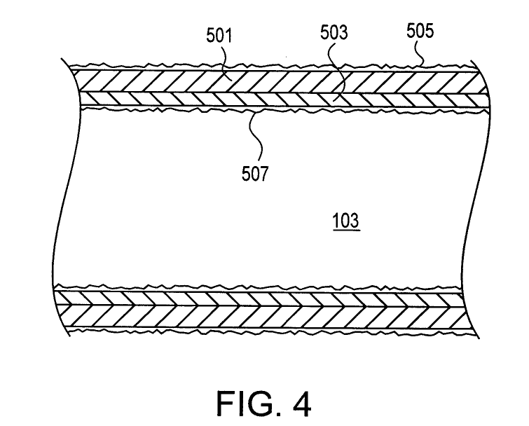

With reference to FIG. 4 the cannula shaft 103 preferably includes an outer cylindrical layer 501 and an inner cylindrical liner 503. The outer cylindrical layer is preferably composed of metal, such as is typically used for a cannula. Alternatively, the outer cylindrical layer may be made from a plastic, a graphite material, a ceramic material, or a carbon fiber material. The inner cylindrical liner is preferably composed of a material having material properties such that the liner becomes destroyed if the cannula shaft is heated in an autoclave at temperatures necessary to sterilize medical instruments. As a result, if the cannula is autoclaved (i.e., if it is placed in an autoclave and heated to a temperature appropriate for sterilizing medical instruments), the cannula becomes unusable. Thus, the cannula will safely be a single use, disposable unit that cannot be reused.

The handle shaft similarly includes an outer cylindrical layer and an inner cylindrical liner, rendering the handle unusable if it is autoclaved. Alternatively, the handle shaft may be made of a single material having material properties such that it becomes destroyed if the handle is heated in an autoclave at temperatures necessary to sterilize medical instruments. Preferably, the handle shaft is composed of a strong plastic to minimize both weight and manufacturing cost.

Preferably, the outer surface of the cannula shaft is coted with a layer of external lubricant 505. This coating of external lubricant is preferably placed on the outer surface of the cannula shaft during the manufacture of the cannula, and prior to the cannula being placed in sterile packaging for delivery to a final user. Preferably, the external lubricant is a polymer coating. Alternatively, the external lubricant may be of a type that is typically applied to a cannula by a physician immediately prior to using the cannula. As another alternative, a cannula user could apply the lubricant by hand as is presently known for traditional lubricants. Optionally, the external lubricant may include a disinfectant.

Preferably, the inner surface of the cannula shaft, which is the an inner surface of the liner for embodiments including a liner, is coated with a layer of internal lubricant 507. The internal lubricant may be of the same type as the external lubricant, or maybe of a different type. The internal lubricant is preferably placed on the inner surface of the cannula shaft during manufacture of the cannula, and prior to the cannula being placed in sterile packaging for delivery to a final user. Preferably, the internal lubricant is a polymer, and forms a polymer coating on the inner surface of the cannula shaft. Optionally, the internal lubricant may include a disinfectant, and/or cell culture materials. The layer of internal lubricant protects withdrawn fluids from contact with the internal surface of the cannula shaft. This layer of internal lubricant also smoothes the flow of fluid along the channel, further protecting the fluid from damage. And inner surface of the handle shaft (and the adjoining passageways) is similarly coated with a layer of internal lubricant.

In a variation of the first embodiment, the handle adapter may be configured identically to the cannula adapter. As such, the handle adapter is configured to be received on a syringe (or other vacuum device) receptacle having the same configuration as the handle receptacle (e.g., a LUER LOK® receptacle).

In a second variation of the first embodiment, the handle adapter may be configured with a typical adapter for reception of a 60 cc syringe.

With reference to FIG. 1, the shaft includes an outer portion forming a grip 551 (i.e., a portion of the shaft that is substantially radially facing and thereby configured for a medical practitioner to grasp the handle by radially compressing the grip within the practitioner's hand. The grip may be composed of the same material as the remainder of the shaft (e.g., plastic), or it may be composed of a separate coating selected to aid a medical practitioner in maintaining their grasp on the handle (e.g., rubber). Preferably, part or all of the grip is larger than ⅞ of an inch in diameter, and more preferably, as large as, or larger than, 1 inch in diameter. As a result, a medical practitioner can maintain a grip on the handle without using excessive hand strength.

The shaft and grip are preferably shaped to minimize fatigue and strain in the hand and arm of a medical practitioner, by providing one or more supports that aid in carrying longitudinal cannula forces to a medical practitioner's hand without the medical practitioner using excessive hand strength. In the first embodiment, the supports include a circumferential flange 553 (i.e., flange extending radially outward around the circumference of the handle) at the distal end of the handle. The circumferential flange extends radially outward far enough for a medical practitioner's thumb and index finger to press longitudinally into the flange when the medical practitioner attempts to thrust the cannula longitudinally forward. More particularly, the circumferential flange preferably extends radially outward to a distance wherein a user's finger and/or thumb bones are longitudinally aligned with a longitudinally facing surface of the flange.

The supports also include a conical ramp extending longitudinally along the grip, being smaller in diameter at the distal end of the grip than at the proximal end of the grip. The conical ramp is sized such that a medical practitioners palm, fingers and thumb provide significant longitudinal support while in a relaxed grip when the medical practitioner attempts to pull the cannula longitudinally back.

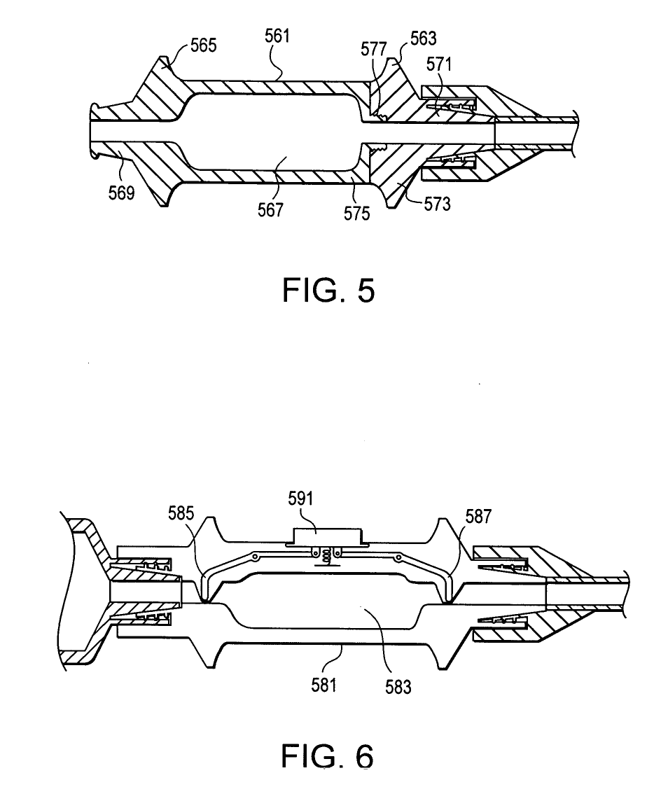

With reference to FIG. 5, in a second embodiment of a handle 561 under the present invention, the handle includes supports, being a first circumferential flange 563 at a distal end of the handle, and a second circumferential flange 565 at a proximal end of the handle. The circumferential flanges preferably extend radially outward to a distance wherein a user's finger and/or thumb bones are longitudinally aligned with the flanges (e.g., ½ inch outward from the portion of the grip contacted by the inward facing portions of the fingers).

The second embodiment is further configured to form a tissue storage chamber. The tissue storage chamber is preferably formed within a shaft channel 567 that connects a passageway of a handle adapter 569 at the proximal end of the handle to a passageway of a handle receptacle 571 at the distal end of the handle, similar to that of the first embodiment. Optionally, the shaft channel may include a portion of increased diameter (with respect to the handle adaptor and handle receptacle passageways), thereby providing for a tissue storage chamber having greater capacity. This feature may take advantage of the available space within some preferred embodiments the handle, which is greater than is found in typical cannula handles.

Preferably, the handle is formed of two portions, e.g., a distal portion 573 including the handle receptacle 571, and a proximal portion 575 including the handle adaptor 569, that threadedly attach to each other with threads 577 to form a fluid tight seal. The two handle portions are configured such that they may be unattached (e.g., unscrewed) to access tissue within the tissue storage chamber.

With reference to FIG. 6, a third embodiment of a handle 581 under the invention also includes a channel 583 that forms a tissue storage chamber having an increased diameter for greater capacity. The channel is configured with two channel closures, a proximal closure 585 and a distal closure 587, each closure being located on opposite longitudinal ends (i.e., a proximal end and a distal end) of the tissue storage chamber. The channel closures are configured to constrict the channel, and may be used to close the channel, and thereby seal tissue within the tissue storage chamber.

Optionally, the closures may be simultaneously actuated by a single trigger mechanism. The trigger mechanism could include a trigger 591, which could be located for use by various fingers such as the second finger (as depicted) or the index finger (not depicted) of a hand holding the handle. Alternatively, the trigger could be located for use by the medical practitioners free hand. This trigger mechanism provides not only for tissue capture and storage within the tissue storage chamber, but also provides for the medical practitioner to have easy on-off control over the suction of tissue through the cannula.

The trigger mechanism may be configured to spring into the closed position (as depicted) when the trigger is not held. Alternatively, the trigger mechanism may be configured to remain in either the open or closed position until it is actively actuated to the other position. This latter configuration is particularly appropriate for use with a trigger mechanism that is actuated by the medical practitioners free hand.

In an alternative embodiment, separate closure actuators may be operated for each closure, such as by mechanisms at each end of the handle.

The first embodiment described above may also optionally be configured with a tissue storage chamber, channel closures, one or more trigger mechanisms, and/or a configuration in which two portions may become unattached to access tissue within the tissue storage chamber. Moreover, each embodiment may be configured with a variety of different handle adaptor types. For example, FIG. 6 depicts a handle adaptor configured for use with a syringe, and using a connection system identical to that described for the cannula-handle connection.

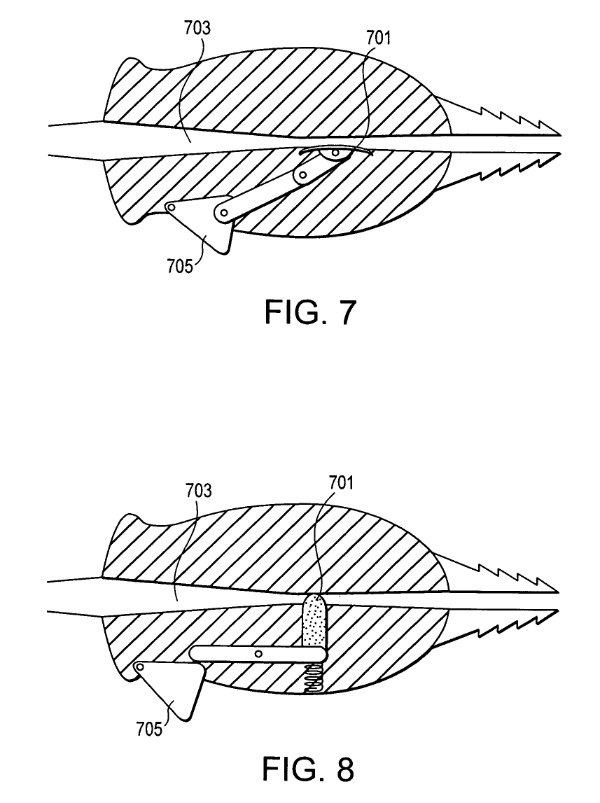

With reference to FIGS. 7 and 8, variations of a fourth embodiment of the invention are configured with a single channel closure 701. The channel closure is configured to constrict a channel 703 within the handle, and may be used to close the channel, and thereby prevent vacuum source vacuum from sucking material through the cannula. As noted with respect to the previous embodiment, the closure may be actuated by a trigger mechanism, including a trigger 705, which could be located for use by various fingers such as the index finger (as depicted) of a hand holding the handle. Alternatively, the trigger could be located for use by the medical practitioners free hand. This trigger mechanism provides for the medical practitioner to have easy on-off control over the suction of tissue through the cannula.

The trigger mechanism may be configured to spring into the closed position (as depicted) when the trigger is not held. Alternatively, the trigger mechanism may be configured to remain in either the open or closed position until it is actively actuated to the other position. This latter configuration is particularly appropriate for use with a trigger mechanism that is actuated by the medical practitioners free hand.

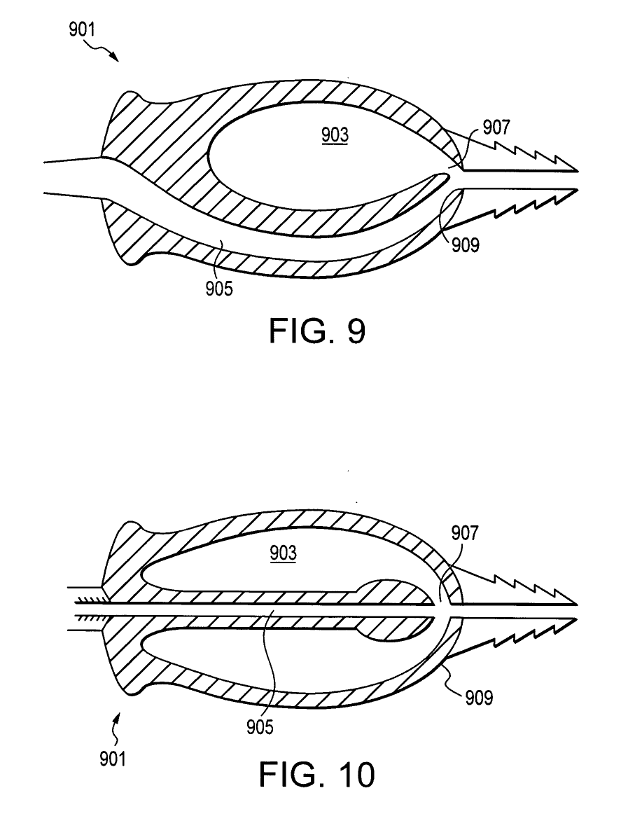

With reference to FIGS. 9 and 10, two variations of a fifth embodiment of a handle 901 of the invention are configured with a storage chamber 903 and a nozzle 905. More particularly, the nozzle extends the channel across the handle without being in free fluid communication with the storage chamber. An opening 907 at a proximal end 909 of the handle provides a flow path between the channel and the storage chamber. As a result, the storage chamber forms a form of Helmholtz resonator, and is configured to regulate the flow of tissue through the handle. As such, tissue can flow into and out of the storage chamber as needed to smooth out the flow of fluid. With the use of this handle (as compared with using a traditional device), a user might experience less hand fatigue, a patient might be subject to less bruising or other damage from a jerking cannula, and the tissue itself will undergo less violent variations in flow rate. Also, sudden discontinuities in vacuum pressure will be smoothed out by vacuum developed and maintained in the storage chamber.

Other features previously described as being used with a storage chamber, such as having one or two trigger operated closures, or having separable handle portions used for accessing the chamber, may also be used with the present embodiment.

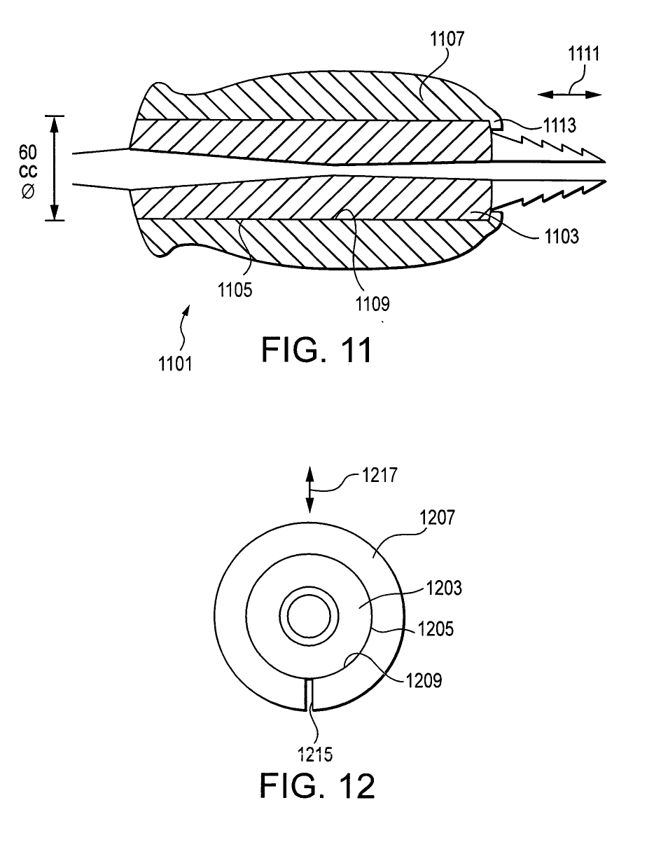

With reference to FIG. 11, two variations of the sixth embodiment of a handle 1101 are configured with a removable grip. In the first variation, the handle includes a handle body 1103, which preferably has a cylindrical outer surface 1105 that is the same size as a commonly-used syringe barrel (e.g., a 35 cc or 60 cc syringe barrel). The handle further includes a removable grip 1107 in the form of a body having a continuous cylindrical inner surface 1109 (i.e., a surface unbroken around the circumference of the grip) that conforms to the cylindrical outer surface of the handle body, providing for a strong friction fit between the grip and the body, and particularly when compressed within the grasp of a user. The grip slides on to a handle body longitudinally 1111. Optionally, the grip or the handle body can have a catch 1113 (such as an end portion) to limit the longitudinal travel of the grip over the handle body.

With reference to FIG. 12 (a longitudinal end view), and using numbers comparable to those of FIG. 11 (incremented by 100), in a second variation, the sixth embodiment includes a grip 1207 having a longitudinal break 1215 (e.g., a cut over the length of the grip) in the grip, thus allowing the grip to applied radially 1217 to the outer surface 1205 handle body 1203 by opening the cylindrical inner surface 1209 of the grip along the longitudinal break to slide over the handle. The grip is formed to be flexible enough to open and radially slide on to the handle body, and then to act as a flexible grip on the handle body. Like the first variation, the second variation may be used on a syringe barrel having the same diameter as a handle body for which the grip is designed (e.g., a 35 cc or 60 cc syringe barrel).

For the second variation, the handle body or syringe barrel need not be cylindrical. Moreover, it is possible for the handle body or syringe barrel to be non-cylindrical, such that the grip is more thoroughly held from longitudinal slipping on the handle body.

Advantageously, what this embodiment provides a handle grip that is usable in a variety of functions, and with a variety of devices. With either variation, the grip can be removed from the handle body and reused, such as on another handle body, or even on a syringe having the same barrel diameter as the handle body. Optionally, such a switch between a handle body and a syringe can be made on-the-fly during a single procedure. Moreover, the grip can be used on a handle body embodying other aspects of the invention. To that end, the grip may include one or more cutouts or semi-hollow portions (i.e., internal hollows configured to not compress a trigger, but to allow a compressive area that can be compressed by a finger to indirectly compress a trigger), and thereby allow compressive access to one or more triggers for one or more channel closures.

It is to be understood that the invention comprises related apparatus and methods for producing cannula and cannula-syringe systems, as well as the apparatus and methods of use for the cannula itself. The above disclosed features can be combined in a wide variety of configurations within the anticipated scope of the invention.

While particular forms of the invention have been illustrated and described, it will be apparent that various modifications can be made without departing from the spirit and scope of the invention. Thus, although the invention has been described in detail with reference only to the preferred embodiments, those having ordinary skill in the art will appreciate that various modifications can be made without departing from the scope of the invention. Accordingly, the invention is not intended to be limited by the above discussion, and is defined with reference to the following claims.

What is claimed is:

1. A handle for use with a cannula forming a cannula channel, and a vacuum source forming a vacuum-source channel, the vacuum source having a receptacle including a hub with an outside cylindrical surface and a concentric threaded inside cylindrical surface, and having a post with a tapered outer surface concentric within the hub, the post forming a passageway concentrically within the post, the passageway forming an opening at an outer end of the post, and the passageway being in fluid communication with the vacuum-source channel, comprising:
a connector configured for connection to the cannula;
a shaft forming a handle channel, the shaft and connector being configured to place the handle channel in fluid communication with the cannula channel; and
a fitting attached to the shaft;
wherein the fitting includes an end portion forming a tapered bore in fluid communication with the handle channel, the tapered bore leading to an end of the shaft;
wherein the fitting end portion further forms a concentric outer threaded surface configured for threadedly receiving the threaded inside cylindrical surface of the hub;
wherein the tapered bore is configured to conformingly receive the tapered outer surface of the receptacle post as the fitting outer threaded surface threadedly receives the threaded inside cylindrical surface of the hub; and
wherein the tapered bore is sized to allow the outer end of the post to compressively contact the end of the shaft.

2. The handle of claim 1, wherein the fitting further includes an elastic sealing device configured to be compressed between the fitting and the receptacle when the receptacle post is received within the tapered bore.

3. A handle for use with a cannula forming a channel, and a vacuum source, comprising:
a connector configured for connection to the cannula;
a shaft having an inner surface forming a handle channel, the shaft and connector being configured to place the handle channel in fluid communication with the cannula channel;
a fitting attached to the shaft, the shaft and fitting being configured to place the handle channel in fluid communication with the vacuum source; and
a layer of lubricant covering the inner surface of the shaft.

4. A handle for use with a cannula forming a channel, and a vacuum source, comprising:
a connector configured for connection to the cannula;
a shaft having an inner layer forming a handle channel, the shaft and connector being configured to place the handle channel in fluid communication with the cannula channel; and
a fitting attached to the shaft, the shaft and fitting being configured to place the handle channel in fluid communication with the vacuum source;
wherein the shaft inner layer is composed of a material that will cause the inner layer to be destroyed when autoclaved.

5. A handle for use with a cannula forming a channel, and a vacuum source, comprising:
a connector configured for connection to the cannula;
a shaft forming a handle channel, the shaft and connector being configured to place the handle channel in fluid communication with the cannula channel; and
a fitting attached to the shaft, the shaft and fitting being configured to place the handle channel in fluid communication with the vacuum source;
wherein at least one of the connector, the shaft and the fitting is composed of a material that will significantly deform when autoclaved.

6. A handle for use with a cannula forming a channel, and a vacuum source, comprising:
a shaft having an inner surface forming a handle channel;
a distal connector on a distal end of the shaft, the distal connector being configured for connection to the cannula; and
a proximal connector on a proximal end of the shaft, the proximal connector being configured for connection to the vacuum source, wherein the shaft, distal connector and proximal connector are configured to place the cannula channel in fluid communication with the vacuum source via the handle channel;
wherein the shaft forms a circumferential surface for grasping by a user, the circumferential surface having a diameter of at least one inch.

7. A handle for use with a cannula forming a channel, and a vacuum source, comprising:
a shaft having an inner surface forming a handle channel;
a distal connector on a distal end of the shaft, the distal connector being configured for connection to the cannula; and
a proximal connector on a proximal end of the shaft, the proximal connector being configured for connection to the vacuum source, wherein the shaft, distal connector and proximal connector are configured to place the cannula channel in fluid communication with the vacuum source via the handle channel;
wherein the shaft forms a grip configured for grasping by a user, and further forms a first circumferential flange extending radially outward from the grip by at least one-half inch.

8. The handle of claim 7, wherein:
the first circumferential flange extends radially outward from the grip at a distal end of the handle by at least one-half inch; and
the shaft forms a second circumferential flange extending radially outward from the grip at a proximal end of the handle by at least one-half inch.

9. A handle for use with a cannula forming a channel, and a vacuum source, comprising:
a shaft having an inner surface forming a tissue storage chamber;
a first connector forming a first passageway, the first connector being configured for connection to the cannula; and
a second connector forming a second passageway, the second connector being configured for connection to the vacuum source, wherein the shaft, the first connector and the second connector are configured to place the cannula channel in fluid communication with the vacuum source via the tissue storage chamber.

10. The handle of claim 9, wherein the tissue storage chamber includes a portion having a diameter larger than that of the first and second passageways.

11. The handle of claim 9, wherein the handle is formed of a distal portion including the first connector and a proximal portion including the second connector, the first and second portions being separably attached, and the first and second portions being detachable to provide direct access to the tissue storage chamber.

12. The handle of claim 9, and further comprising a first channel closure configured to seal tissue within the tissue storage chamber with respect to at least one of the cannula and the vacuum source.

13. The handle of claim 12, and further comprising a second channel closure wherein the first and second channel closures are configured to seal tissue within the tissue storage chamber with respect to both the cannula and the vacuum source.

14. A handle for use with a cannula forming a channel, and a vacuum source, comprising:
a body having a first inner surface forming a tissue storage chamber and a second inner surface forming a channel that bypasses the tissue storage chamber;
a first connector forming a first passageway, the first connector being configured for connection to the cannula; and
a second connector forming a second passageway, the second connector being configured for connection to the vacuum source, wherein the channel places the first and second passageways in fluid communication, and wherein the tissue storage chamber opens into the channel.

15. A handle for use with a cannula forming a channel, and a vacuum source, comprising:
a handle body having an inner surface forming a handle channel;
a distal connector on a distal end of the handle body, the distal connector being configured for connection to the cannula;
a proximal connector on a proximal end of the handle body, the proximal connector being configured for connection to the vacuum source, wherein the handle body, distal connector and proximal connector are configured to place the cannula channel in fluid communication with the vacuum source via the handle channel; and
a removable grip that is conformingly received over the handle body.

16. The handle of claim 15, wherein the handle body includes a reception surface that conformingly receives the grip, and wherein the reception surface is a cylinder that is substantially the same size as either a 60 cc syringe or a 35 cc syringe.
